(19) United States
(12) Patent Application Publication
(10) Pub. No.: US 2021/0017266 A1
(43) Pub. Date: Jan. 21, 2021

(54) METHODS FOR TREATING OCULAR DISEASES

(71) Applicant: Novartis AG, Basel (CH)

(72) Inventors: Amy RACINE, Birsfelden (CH); James WARBURTON, Buckinghamshire (GB); Andreas WEICHSELBERGER, Basel (CH)

(21) Appl. No.: 16/980,679

(22) PCT Filed: Mar. 8, 2019
(86) PCT No.: PCT/IB2019/051899

§ 371 Date: Sep. 14, 2020

Related U.S. Application Data
(60) Provisional application No. 62/643,887, filed on Mar. 16, 2018.
(60) Provisional application No. 62/805,344, filed on Feb. 14, 2019.

(51) Int. Cl. C07K 16/22; A61P 27/06

(52) U.S. Cl. CPC C07K 16/22; A61P 27/06; A61K 2039/545; C07K 2317/622; C07K 2317/76; C07K 2317/24

(57) ABSTRACT

A method is provided for treating a patient having a neovascular ocular disease.

### FIELD OF THE INVENTION

The invention relates to methods for treating ocular disease with a VEGF antagonist. In particular, the invention relates to treating diabetic macular edema with less frequent dosing than currently approved treatment regimens.

### BACKGROUND OF THE INVENTION

Diabetes mellitus (DM) is the most common endocrine disease in developed countries, with prevalence estimates ranging between 2 to 5% of the world population. Diabetic retinopathy (DR) and diabetic macular edema (DME) are common microvascular complications in patients with diabetes and may have a debilitating impact on visual acuity (VA), eventually leading to blindness. DME is a frequent manifestation of DR (Riordan-Eva, 2004, Eye (Lond). 2004, 18:1161-8) and is the major cause of visual loss in patients with DR.

For anti-VEGF agents like ranibizumab or aflibercept a favorable benefit risk ratio was demonstrated with superior efficacy versus the previous standard of care (laser photocoagulation) in large Phase 3 programs that consequently led to their approval for the treatment of DME. Anti-VEGF treatment led to clinically relevant improvements of BCVA, reduction of fluid accumulation and decreased severity of diabetic retinopathy.

The current treatment options for patients with DME are: laser photocoagulation, intravitreal (IVT) corticosteroids, IVT corticosteroid implants, or IVT anti-VEGF therapeutic. Due to the efficacy and safety profile of anti-VEGF therapy, it has become the first-line treatment. Corticosteroids are used as a second line treatment and focal/grid laser photocoagulation remains a therapeutic option, but with a lower expected benefit compared with steroid and anti-VEGF therapy.

Despite the treatment success of existing anti-VEGFs, there remains a need for further treatment options to improve response rate and/or reduce resource use and injection frequency in patients with DME (Mitchell et al., 2011, Ophthalmology 118(4):615-25; Smiddy, 2011, Ophthalmology 118(9):1827-33; Lang et al., 2013, Ophthalmology 120(10):2004-12; Virgili et al., 2014, Br J Ophthalmol 98(4):421-2; Agarwal et al., 2015, Curr Diab Rep. 15(10):75).

### SUMMARY

The invention provides an improved method of administering a therapeutic VEGF antagonist for treating ocular diseases, in particular diabetic macular edema (DME). In certain aspects, the invention provides methods for treating DME comprising administering to a mammal five individual doses of a VEGF antagonist at 6-week intervals, followed by additional doses every 12 weeks (q12) and/or every 8 weeks (q8) depending on the outcome of disease activity assessments using pre-defined visual and anatomic criteria. In one aspect, dosing frequency can be extended four more weeks if disease activity is not detected at certain scheduled treatment visits.

The invention also provides a VEGF antagonist for use in a method of treating ocular diseases, particularly ocular neovascular diseases, more particularly diabetic macular edema (DME), in a patient, wherein the VEGF antagonist is first provided in a loading phase, during which the patient receives five individual doses of the VEGF antagonist at 6-week intervals, and then the VEGF antagonist is provided in a maintenance phase, during which the patient receives an additional dose of the VEGF antagonist once every 8 weeks (q8w regimen) or once every 12 weeks (q12w regimen).

In certain aspects, the VEGF antagonist used in a method of the invention is an anti-VEGF antibody. In a particular aspect, the anti-VEGF antibody is a single chain antibody (scFv) or Fab fragment. In particular, the anti-VEGF antibody is RTH258.

Specific preferred embodiments of the invention will become evident from the following more detailed description of certain preferred embodiments and the claims.

### DETAILED DESCRIPTION

### Definitions

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary, 3rd Edition or a dictionary known to those of skill in the art, such as the Oxford Dictionary of Biochemistry and Molecular Biology (Ed. Anthony Smith, Oxford University Press, Oxford, 2004).

As used herein, all percentages are percentages by weight, unless stated otherwise.

As used herein and unless otherwise indicated, the terms “a” and “an” are taken to mean “one”, “at least one” or “one or more”. Unless otherwise required by context, singular terms used herein shall include pluralities and plural terms shall include the singular.

The contents of any patents, patent applications, and references cited throughout this specification are hereby incorporated by reference in their entireties.

The term “VEGF” refers to the 165-amino acid vascular endothelial cell growth factor, and related 121-, 189-, and 206-amino acid vascular endothelial cell growth factors, as described by Leung et al., Science 246:1306 (1989), and Houck et al., Mol. Endocrin. 5:1806 (1991) together with the naturally occurring allelic and processed forms of those growth factors.

The term “VEGF receptor” or “VEGFr” refers to a cellular receptor for VEGF, ordinarily a cell-surface receptor found on vascular endothelial cells, as well as variants thereof retaining the ability to bind hVEGF. One example of a VEGF receptor is the fms-like tyrosine kinase (flt), a transmembrane receptor in the tyrosine kinase family. DeVries et al., Science 255:989 (1992); Shibuya et al., Oncogene 5:519 (1990). The flt receptor comprises an extracellular domain, a transmembrane domain, and an intracellular domain with tyrosine kinase activity. The extracellular domain is involved in the binding of VEGF, whereas the intracellular domain is involved in signal transduction. Another example of a VEGF receptor is the flk-1 receptor (also referred to as KDR). Matthews et al., Proc. Nat. Acad. Sci. 88:9026 (1991); Terman et al., Oncogene 6:1677 (1991); Terman et al., Biochem. Biophys. Res. Commun. 187:1579 (1992). Binding of VEGF to the flt receptor results in the formation of at least two high molecular weight complexes, having an apparent molecular weight of 205,000 and 300,000 Daltons. The 300,000 Dalton complex is believed to be a dimer comprising two receptor molecules bound to a single molecule of VEGF.

As used herein, a “VEGF antagonist” refers to a compound that can diminish or inhibit VEGF activity in vivo. A VEGF antagonist can bind to a VEGF receptor(s) or block VEGF protein(s) from binding to VEGF receptor(s). A VEGF antagonist can be, for example, a small molecule, an anti-VEGF antibody or antigen-binding fragments thereof, fusion protein (such as aflibercept or other such soluble decoy receptor), an aptamer, an antisense nucleic acid molecule, an interfering RNA, receptor proteins, and the like that can bind specifically to one or more VEGF proteins or one or more VEGF receptors. Several VEGF antagonists are described in WO 2006/047325.

In a preferred embodiment, the VEGF antagonist is an anti-VEGF antibody (such as RTH258 or ranibizumab) or a soluble VEGF receptor (such as aflibercept).

The term “antibody” as used herein includes whole antibodies and any antigen binding fragment (i.e., “antigen-binding portion,” “antigen binding polypeptide,” or “immunobinder”) or single chain thereof. An “antibody” includes a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “single chain antibody”, “single chain Fv” or “scFv” is intended to refer to a molecule comprising an antibody heavy chain variable domain (or region; V_{H}) and an antibody light chain variable domain (or region; V_{L}) connected by a linker. Such scFv molecules can have the general structures: NH₂-V_{L}-linker-V_{H}—COOH or NH₂-V_{H}-linker-V_{L}-COOH.

The term “antigen-binding portion” of an antibody (or simply “antibody portion”) refers to one or more fragments of an antibody that retain the ability to specifically bind to an antigen (e.g., VEGF). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a single domain or dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins. Antibodies can be of different isotype, for example, an IgG (e.g., an IgG1, IgG2, IgG3, or IgG4 subtype), IgA1, IgA2, IgD, IgE, or IgM antibody.

As used herein, a “mammal” includes any animal classified as a mammal, including, but not limited to, humans, domestic animals, farm animals, and companion animals, etc.

As used herein, the term “subject” or “patient” refers to human and non-human mammals, including but, not limited to, primates, pigs, horses, dogs, cats, sheep, and cows. Preferably, a subject or patient is a human.

An “ocular disease” or “neovascular ocular disease” that can be treated using a method of the invention includes, a condition, disease, or disorder associated with ocular neovascularization, including, but not limited to, abnormal angiogenesis, choroidal neovascularization (CNV), retinal vascular permeability, retinal edema, diabetic retinopathy (particularly proliferative diabetic retinopathy), diabetic macular edema (DME), neovascular (exudative) age-related macular degeneration (AMD), including CNV associated with nAMD (neovascular AMD), sequela associated with retinal ischemia, Central Retinal Vein Occlusion (CRVO), Branch Retinal Vein Occlusion (BRVO), and posterior segment neovascularization. In a preferred embodiment, the disease is DME. In certain embodiments, the disease is macular edema secondary to CRVO or BRVO.

### Treatment Regimen

The invention provides methods for determining whether patients being treated with a VEGF antagonist for an ocular disease can be treated every eight weeks or every twelve weeks or every 16 weeks.

The invention provides methods for treating ocular neovascular diseases, including DME, in a mammal, the methods comprising administering multiple doses of a VEGF antagonist to the mammal at various intervals for at least two years. In certain embodiments, the doses are administered at five 6-week intervals, the “loading phase,” followed by administering additional doses at 8-week, 9-week, 10-week, 11-week, or 12-week intervals (i.e., q12w) during the “maintenance phase.” Disease activity assessments are conducted at least at every additional scheduled administration during the maintenance phase. When disease activity is identified as described herein, the treatment regimen is changed from every 12 weeks to every 8 weeks (i.e., q8w). The invention provides specific criteria established by the inventors based on disease activity assessments to determine when an 8-week interval should be used and when a 12-week interval should be continued. In some cases, a patient might be on a 12-week interval regimen for some time, and then switch to an 8-week interval, and then switch back to the 12-week interval. Thus, patients may not stay on one interval regimen, and may go back and forth depending on assessments according to the criteria set forth herein.

In one embodiment, when disease activity is not detected for multiple consecutive treatment visits, the treatment provider can extend treatment an additional one to four weeks. For example, if a patient is being treated every 12 weeks, the treatment provider may extend treatments to every 13, 14, 15, or 16 weeks; or if a patient is being treated every 8 weeks, the treatment provider may extend treatments to every 9, 10, 11, or 12 weeks. If disease activity is identified at any treatment visit, the treatment schedule is adjusted back to the 12 week or 8 week treatment regimen. As used herein, “disease activity” refers to worsening of the ocular disease based on criteria provided herein.

In one embodiment, the invention provides a method for treating ocular diseases, particularly ocular neovascular diseases, more particularly DME, comprising administering a VEGF antagonist to a mammal in need thereof according to the following schedule:
a “loading phase” of 5 doses administered at 6-week (i.e., “q6” or “q6w”) intervals (e.g., day 0, week 6, week 12, week 18, week 24), and
a “maintenance phase” of additional doses administered at 12-week (i.e., “q12” or “q12w”) intervals.

In certain embodiments, the “maintenance phase” can be additional doses at 8, 9, 10, 11, 12, 13, 14, 15, or 16 week intervals, and can be adjusted as described herein based on Disease Activity Assessments as described herein.

In certain embodiments, the “loading phase” can be 5 doses administered at 4-week (q4w) or q6w intervals or 4 doses administered at q4w or q6w intervals. In certain embodiments, where the ocular disease to be treated is BRVO or CRVO (e.g., macular edema secondary to BRVO or CRVO) the loading phase is 4 doses or 5 doses at q4w intervals followed by a maintenance phase as described above and herein.

In certain embodiments, a Disease Activity Assessment (“DAA”) is conducted at all scheduled treatment visits. In one embodiment, a patient is reassigned to q8 dosing regimen based on the presence of certain level of disease activity as determined by a treatment provider.

At assessment weeks, the patients can be currently on an 8-week or 12-week interval regimen. Thus, the assessment can determine if a patient stays on the current interval or switches to the other interval.

An assessment as described herein preferably includes one or more of the following tests to assess activity of RTH258 on visual function, retinal structure and leakage:
Best-corrected visual acuity with ETDRS-like chart at 4 meters
Anatomical markers on Optical Coherence Tomography
ETDRS DRSS score based on 7-field stereo Color Fundus Photography
Vascular leakage evaluation by Fluorescein Angiography

Visual acuity can be assessed using best correction determined from protocol refraction (BCVA). BCVA measurements can be taken in a sitting position using ETDRS—like visual acuity testing charts.

Optical Coherence Tomography (OCT), color fundus photography and fluorescein angiography can be assessed according to methods known to those of skill in the art.

Additional criteria for assessing disease activity includes, but is not limited to, changes in central subfield thickness (CST). The CST is the average thickness of circular 1 mm area centered around the fovea measured from retinal pigment epithelium (RPE) to the internal limiting membrane (ILM), inclusively. CST can be measured, for example, using spectral domain Optical Coherence Tomography (SD-OCT).

Means of performing the above tests are well understood and commonly used by those skilled in the art.

Disease activity is assessed for clinically relevant improvements of BCVA, reduction of central subfield thickness (CST), reduction of fluid accumulation (e.g., retinal fluid) and/or decreased severity of diabetic retinopathy. Where disease activity is worsening (for example, loss of letters measured by BCVA, increase in CST, increased fluid accumulation, and or increased severity of diabetic retinopathy), a more frequent dosing interval is prescribed going forward. Where improvement of disease activity is observed, a less frequent dosing interval is prescribed. Where there is neither worsening nor improvement of disease activity, the dosing interval is maintained or extended (less frequent). Fluid measured in the eye can be intraretinal and/or subretinal fluid.

Assessing status of disease activity can be based, for example, on dynamic changes in BCVA, central subfield thickness (CST), and/or intraretinal fluid status assessed, for example, by spectral domain optical coherence tomography. Thereafter, guidance can be based, for example, on BCVA decline due to disease activity compared with a previous assessment. It should be understood the treating clinician can make a decision based on clinical judgment, which can include more than visual acuity criteria. Disease activity assessments can include both visual acuity and anatomical criteria.

In one embodiment, assessments of DME disease activity to establish the patient's disease status occurs at Week 28 (outcome of the loading treatment). The assessment of the disease activity (DAA) during treatment regimens is at the discretion of the person making the assessment (e.g., the treatment provider), and is based on changes in vision and anatomical parameters with reference to the patients' disease status at Week 28. The outcome of this assessment is captured as:
‘q8w-need’: identified disease activity that according to the treatment provider requires more frequent anti-VEGF treatment, e.g.: ≥5 letters loss in BCVA (compared to Week 28) which, based on anatomical parameters, is attributable to DME disease activity.
‘no q8w-need’: otherwise if DAA reveals a need for more q8w treatment the subject is assigned to receive injections q8w thereafter. If disease status improves, the treatment provider can place the patient back on a q12w treatment schedule.

If DAA reveals a need for more frequent treatment the patient will be assigned to receive injections q8w thereafter, or up to a treatment interval extension based on the stability assessment at Week 72 as described herein.

In certain embodiments, a patient can be treated with brolucizumab once every four weeks (q4w) or once every six weeks (q6w), and a treatment provider can assess disease activity at each treatment or before a scheduled treatment to determine if less frequent dosing (e.g., a q8w or q12w or q16w) schedule is appropriate using, for example, the DAA as described herein. For example, a patient may be on a q4w treatment regimen for several months and then be switched to a less frequent dosing (e.g., q8w, q12w, or q16w) schedule based on a favorable DAA.

### Anti-VEGF Antibodies

In certain embodiments, a VEGF antagonist used in a method of the invention is an anti-VEGF antibody, particularly anti-VEGF antibodies described in WO 2009/155724, the entire contents of which are hereby incorporated by reference.

In one embodiment, the anti-VEGF antibody of the invention comprises a variable heavy chain having the sequence as set forth in SEQ ID NO: 1 and a variable light chain having the sequence as set forth in SEQ ID NO: 2.

VH:
SEQ ID NO. 1
EVQLVESGGGLVQPGGSLRLSCTASGFSLTDYYYMTWVRQAPGKGLEWVG
FIDPDDDPYYATWAKGRFTISRDNSKNTLYLQMNSLRAEDTAVYYCAGGD
HNSGWGLDIWGQGTLVTVSS
VL:
SEQ ID NO. 2
EIVMTQSPSTLSASVGDRVIITCQASEIIHSWLAWYQQKPGKAPKLLIYL
ASTLASGVPSRFSGSGSGAEFTLTISSLQPDDFATYYCQNVYLASTNGAN
FGQGTKLTVLG

In another embodiment, the anti-VEGF antibody used in a method of the invention comprises the sequence as set forth in SEQ ID NO: 3.

EIVMTQSPSTLSASVGDRVIITCQASEIIHSWLAWYQQKPGKAPKLLIYL
ASTLASGVPSRFSGSGSGAEFTLTISSLQPDDFATYYCQNVYLASTNGAN
FGQGTKLTVLGGGGGSGGGGSGGGGSGGGGSEVQLVESGGGLVQPGGSLR
LSCTASGFSLTDYYYMTWVRQAPGKGLEWVGFIDPDDDPYYATWAKGRFT
ISRDNSKNTLYLQMNSLRAEDTAVYYCAGGDHNSGWGLDIWGQGTLVTVS
S

In a preferred embodiment, the anti-VEGF antibody used in a method of the invention is RTH258 (which comprises SEQ ID NO: 3). A methionine derived from the start codon in an expression vector is present in the final protein in cases where it has not been cleaved posttranslationally as follows.

(SEQ ID NO: 4)
MEIVMTQSPS TLSASVGDRV IITCQASEII HSWLAWYQQK
PGKAPKLLIY LASTLASGVP SRFSGSGSGA EFTLTISSLQ
PDDFATYYCQ NVYLASTNGA NFGQGTKLTV LGGGGGSGGG
GSGGGGSGGG GSEVQLVESG GGLVQPGGSL RLSCTASGFS
LTDYYYMTWV RQAPGKGLEW VGFIDPDDDP YYATWAKGRF
TISRDNSKNT LYLQMNSLRA EDTAVYYCAG GDHNSGWGLD
IWGQGTLVTV SS

RTH258, also known as brolucizumab, is a humanized single-chain FIT (scFv) antibody fragment inhibitor of VEGF with a molecular weight of −26 kDa. It is an inhibitor of VEGF-A and works by binding to the receptor binding site of the VEGF-A molecule, thereby preventing the interaction of VEGF-A with its receptors VEGFR1 and VEGFR2 on the surface of endothelial cells. Increased levels of signaling through the VEGF pathway are associated with pathologic ocular angiogenesis and retinal edema. Inhibition of the VEGF pathway has been shown to inhibit the growth of neovascular lesions and resolve retinal edema in patients with nAMD.

### Pharmaceutical Preparations

In one aspect the methods of the invention comprise the use of pharmaceutical formulations comprising anti-VEGF antibodies. The term “pharmaceutical formulation” refers to preparations which are in such form as to permit the biological activity of the antibody or antibody derivative to be unequivocally effective, and which contain no additional components which are toxic to the subjects to which the formulation would be administered. “Pharmaceutically acceptable” excipients (vehicles, additives) are those which can reasonably be administered to a subject mammal to provide an effective dose of the active ingredient employed.

A “stable” formulation is one in which an antibody or antibody derivative therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10: 29-90 (1993), for example. Stability can be measured at a selected temperature for a selected time period. Preferably, the formulation is stable at room temperature (about 30° C.) or at 40° C. for at least 1 week and/or stable at about 2-8° C. for at least 3 months to 2 years. Furthermore, the formulation is preferably stable following freezing (to, e.g., −70° C.) and thawing of the formulation.

An antibody or antibody derivative “retains its physical stability” in a pharmaceutical formulation if it meets the defined release specifications for aggregation, degradation, precipitation and/or denaturation upon visual examination of color and/or clarity, or as measured by UV light scattering or by size exclusion chromatography, or other suitable art recognized methods.

An antibody or antibody derivative “retains its chemical stability” in a pharmaceutical formulation, if the chemical stability at a given time is such that the protein is considered to still retain its biological activity as defined below. Chemical stability can be assessed by detecting and quantifying chemically altered forms of the protein. Chemical alteration may involve size modification (e.g. clipping) which can be evaluated using size exclusion chromatography, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example.

An antibody or antibody derivative “retains its biological activity” in a pharmaceutical formulation, if the biological activity of the antibody at a given time is within about 10% (within the errors of the assay) of the biological activity exhibited at the time the pharmaceutical formulation was prepared as determined in an antigen binding assay, for example. Other “biological activity” assays for antibodies are elaborated herein below.

By “isotonic” is meant that the formulation of interest has essentially the same osmotic pressure as human blood. Isotonic formulations will generally have an osmotic pressure from about 250 to 350 mOsm. Isotonicity can be measured using a vapor pressure or ice-freezing type osmometer, for example.

A “polyol” is a substance with multiple hydroxyl groups, and includes sugars (reducing and non-reducing sugars), sugar alcohols and sugar acids. Preferred polyols herein have a molecular weight which is less than about 600 kD (e.g. in the range from about 120 to about 400 kD). A “reducing sugar” is one which contains a hemiacetal group that can reduce metal ions or react covalently with lysine and other amino groups in proteins and a “non-reducing sugar” is one which does not have these properties of a reducing sugar. Examples of reducing sugars are fructose, mannose, maltose, lactose, arabinose, xylose, ribose, rhamnose, galactose and glucose. Non-reducing sugars include sucrose, trehalose, sorbose, melezitose and raffinose Mannitol, xylitol, erythritol, threitol, sorbitol and glycerol are examples of sugar alcohols. As to sugar acids, these include L-gluconate and metallic salts thereof. Where it is desired that the formulation is freeze-thaw stable, the polyol is preferably one which does not crystallize at freezing temperatures (e.g. −20° C.) such that it destabilizes the antibody in the formulation. Non-reducing sugars such as sucrose and trehalose are the preferred polyols herein, with trehalose being preferred over sucrose, because of the superior solution stability of trehalose.

As used herein, “buffer” refers to a buffered solution that resists changes in pH by the action of its acid-base conjugate components. The buffer of this invention has a pH in the range from about 4.5 to about 8.0; preferably from about 5.5 to about 7. Examples of buffers that will control the pH in this range include acetate (e.g. sodium acetate), succinate (such as sodium succinate), gluconate, histidine, citrate and other organic acid buffers. Where a freeze-thaw stable formulation is desired, the buffer is preferably not phosphate.

In a pharmacological sense, in the context of the present invention, a “therapeutically effective amount” of an antibody or antibody derivative refers to an amount effective in the prevention or treatment of a disorder for the treatment of which the antibody or antibody derivative is effective. A “disease/disorder” is any condition that would benefit from treatment with the antibody or antibody derivative. This includes chronic and acute disorders or diseases including those pathological conditions which predispose the mammal to the disorder in question.

A “preservative” is a compound which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzethonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservative herein is benzyl alcohol.

The pharmaceutical compositions used in present invention comprise a VEGF antagonist, preferably an anti-VEGF antibody (e.g., an anti-VEGF antibody comprising the variable light chain sequence of SEQ ID NO: 1 and the variable heavy chain sequence of SEQ ID NO: 2, such as brolucizumab), together with at least one physiologically acceptable carrier or excipient. Pharmaceutical compositions may comprise, for example, one or more of water, buffers (e.g., neutral buffered saline or phosphate buffered saline), ethanol, mineral oil, vegetable oil, dimethylsulfoxide, carbohydrates (e.g., glucose, mannose, sucrose or dextrans), mannitol, proteins, adjuvants, polypeptides or amino acids such as glycine, antioxidants, chelating agents such as EDTA or glutathione and/or preservatives. As noted above, other active ingredients may (but need not) be included in the pharmaceutical compositions provided herein.

A carrier is a substance that may be associated with an antibody or antibody derivative prior to administration to a patient, often for the purpose of controlling stability or bioavailability of the compound. Carriers for use within such formulations are generally biocompatible, and may also be biodegradable. Carriers include, for example, monovalent or multivalent molecules such as serum albumin (e.g., human or bovine), egg albumin, peptides, polylysine and polysaccharides such as aminodextran and polyamidoamines. Carriers also include solid support materials such as beads and microparticles comprising, for example, polylactate polyglycolate, poly(lactide-co-glycolide), polyacrylate, latex, starch, cellulose or dextran. A carrier may bear the compounds in a variety of ways, including covalent bonding (either directly or via a linker group), noncovalent interaction or admixture.

Pharmaceutical compositions may be formulated for any appropriate manner of administration, including, for example, topical, intraocular, oral, nasal, rectal or parenteral administration. In certain embodiments, compositions in a form suitable for intraocular injection, such as intravitreal injection, are preferred. Other forms include, for example, pills, tablets, troches, lozenges, aqueous or oily suspensions, dispersible powders or granules, emulsion, hard or soft capsules, or syrups or elixirs. Within yet other embodiments, compositions provided herein may be formulated as a lyophilizate. The term parenteral as used herein includes subcutaneous, intradermal, intravascular (e.g., intravenous), intramuscular, spinal, intracranial, intrathecal and intraperitoneal injection, as well as any similar injection or infusion technique.

The pharmaceutical composition may be prepared as a sterile injectible aqueous or oleaginous suspension in which the active agent (i.e. VEGF antagonist), depending on the vehicle and concentration used, is either suspended or dissolved in the vehicle. Such a composition may be formulated according to the known art using suitable dispersing, wetting agents and/or suspending agents such as those mentioned above. Among the acceptable vehicles and solvents that may be employed are water, 1,3-butanediol, Ringer's solution and isotonic sodium chloride solution. In addition, sterile, fixed oils may be employed as a solvent or suspending medium. For this purpose any bland fixed oil may be employed, including synthetic mono- or diglycerides. In addition, fatty acids such as oleic acid may be used in the preparation of injectible compositions, and adjuvants such as local anesthetics, preservatives and/or buffering agents can be dissolved in the vehicle.

### Dosage

A dose used in a method of the invention is based on the specific disease or condition being treated. The term “therapeutically effective dose” is defined as an amount sufficient to achieve or at least partially achieve the desired effect. A therapeutically effective dose is sufficient if it can produce even an incremental change in the symptoms or conditions associated with the disease. The therapeutically effective dose does not have to completely cure the disease or completely eliminate symptoms. Preferably, the therapeutically effective dose can at least partially arrest the disease and its complications in a patient already suffering from the disease. Amounts effective for this use will depend upon the severity of the disorder being treated and the general state of the patient's own immune system.

The dose amount can be readily determined using known dosage adjustment techniques by a physician having ordinary skill in treatment of the disease or condition. The therapeutically effective amount of a VEGF antagonist used in a method of the invention is determined by taking into account the desired dose volumes and mode(s) of administration, for example. Typically, therapeutically effective compositions are administered in a dosage ranging from 0.001 mg/ml to about 200 mg/ml per dose. Preferably, a dosage used in a method of the invention is about 60 mg/ml to about 120 mg/ml (for example, a dosage is 60, 70, 80, 90, 100, 110, or 120 mg/ml). In a preferred embodiment, the dosage of an anti-VEGF antibody used in a method of the invention is 60 mg/ml or 120 mg/ml.

In certain embodiments, a dose is administered directly to an eye of a patient. In one embodiment, a dose per eye is at least about 0.5 mg up to about 6 mg. Preferred doses per eye include about 0.5 mg, 0.6 mg, 0.7 mg, 0.8 mg, 0.9 mg, 1.0 mg, 1.2 mg, 1.4 mg, 1.6 mg, 1.8 mg, 2.0 mg, 2.5 mg, 3.0 mg, 3.5 mg, 4.0 mg, 4.5 mg, 5.0 mg, 5.5 mg, and 6.0 mg. Doses can be administered in various volumes suitable for ophthalmic administration, such as 50 μl or 100 IA, for example, including 3 mg/50 μl or 6 mg/50 μl. Smaller volumes can also be used, including 20 μl or less, for example about 20 μl, about 10 μl, or about 8.0 μl. In certain embodiments, a dose of 2.4 mg/20 μl, 1.2 mg/10 μl or 1 mg/8.0 μl (e.g., 1 mg/8.3 μl) is delivered to an eye of a patient for treating or ameliorating one or more of the diseases and disorders described above. Delivery can be, for example, by intravitreal injection.

As used herein, the term “about” includes and describes the value or parameter per se. For example, “about x” includes and describes “x” per se. As used herein, the term “about” when used in association with a measurement, or used to modify a value, a unit, a constant, or a range of values, refers to variations of ±1-10% in addition to including the value or parameter per se. In some embodiments, the term “about” when used in association with a measurement, or used to modify a value, a unit, a constant, or a range of values, refers to variations of ±1, ±2, ±3, ±4, ±5, ±6, ±7, ±8, ±9, or ±10%.

An aqueous formulation of an anti-VEGF antibody used in a method of the invention is prepared in a pH-buffered solution. Preferably, the buffer of such aqueous formulation has a pH in the range from about 4.5 to about 8.0, preferably from about 5.5 to about 7.0, most preferably about 6.75. In one embodiment, the pH of an aqueous pharmaceutical composition of the invention is about 7.0-7.5, or about 7.0-7.4, about 7.0-7.3, about 7.0-7.2, about 7.1-7.6, about 7.2-7.6, about 7.3-7.6 or about 7.4-7.6. In one embodiment, an aqueous pharmaceutical composition of the invention has a pH of about 7.0, about 7.1, about 7.2, about 7.3, about 7.4, about 7.5 or about 7.6. In a preferred embodiment, the aqueous pharmaceutical composition has a pH of ≥7.0 In a preferred embodiment, the aqueous pharmaceutical composition has a pH of about 7.2. In another preferred embodiment, the aqueous pharmaceutical composition has a pH of about 7.4. In another preferred embodiment, the aqueous pharmaceutical composition has a pH of about 7.6. Examples of buffers that will control the pH within this range include acetate (e.g. sodium acetate), succinate (such as sodium succinate), gluconate, histidine, citrate and other organic acid buffers. The buffer concentration can be from about 1 mM to about 50 mM, preferably from about 5 mM to about 30 mM, depending, for example, on the buffer and the desired isotonicity of the formulation.

A polyol, which acts as a tonicifier, may be used to stabilize an antibody in an aqueous formulation. In preferred embodiments, the polyol is a non-reducing sugar, such as sucrose or trehalose. If desired, the polyol is added to the formulation in an amount that may vary with respect to the desired isotonicity of the formulation. Preferably the aqueous formulation is isotonic, in which case suitable concentrations of the polyol in the formulation are in the range from about 1% to about 15% w/v, preferably in the range from about 2% to about 10% w/v, for example. However, hypertonic or hypotonic formulations may also be suitable. The amount of polyol added may also alter with respect to the molecular weight of the polyol. For example, a lower amount of a monosaccharide (e.g. mannitol) may be added, compared to a disaccharide (such as trehalose).

A surfactant is also added to an aqueous antibody formulation. Exemplary surfactants include nonionic surfactants such as polysorbates (e.g. polysorbates 20, 80 etc) or poloxamers (e.g. poloxamer 188). The amount of surfactant added is such that it reduces aggregation of the formulated antibody/antibody derivative and/or minimizes the formation of particulates in the formulation and/or reduces adsorption. For example, the surfactant may be present in the formulation in an amount from about 0.001% to about 0.5%, preferably from about 0.005% to about 0.2% and most preferably from about 0.01% to about 0.1%.

In one embodiment, an aqueous antibody formulation used in a method of the invention is essentially free of one or more preservatives, such as benzyl alcohol, phenol, m-cresol, chlorobutanol and benzethonium Cl. In another embodiment, a preservative may be included in the formulation, particularly where the formulation is a multidose formulation. The concentration of preservative may be in the range from about 0.1% to about 2%, most preferably from about 0.5% to about 1%. One or more other pharmaceutically acceptable carriers, excipients or stabilizers such as those described in Remington's Pharmaceutical Sciences 21st edition, Osol, A. Ed. (2006) may be included in the formulation provided that they do not adversely affect the desired characteristics of the formulation. Acceptable carriers, excipients or stabilizers are non-toxic to recipients at the dosages and concentrations employed and include: additional buffering agents, co-solvents, antioxidants including ascorbic acid and methionine, chelating agents such as EDTA, metal complexes (e.g. Zn-protein complexes), biodegradable polymers such as polyesters, and/or salt-forming counterions such as sodium.

Formulations to be used for in vivo administration must be sterile. This is readily accomplished by filtration through sterile filtration membranes, prior to, or following, preparation of the formulation.

In one embodiment, a VEGF antagonist is administered to an eye of a mammal in need of treatment in accordance with known methods for ocular delivery. Preferably, the mammal is a human, the VEGF antagonist is an anti-VEGF antibody, and the antibody is administered directly to an eye. Administration to a patient can be accomplished, for example, by intravitreal injection.

The VEGF antagonist in a method of the invention can be administered as the sole treatment or in conjunction with other drugs or therapies useful in treating the condition in question.

A preferred formulation for RTH258 for intravitreal injection comprises about 4.5% to 11% (w/v) sucrose, 5-20 mM sodium citrate, and 0.001% to 0.05% (w/v) polysorbate 80, wherein the pH of the formulation is about 7.0 to about 7.4. One such formulation is shown in the table below. Another such formulation comprises 5.9% (w/v) sucrose, 10 mM sodium citrate, 0.02% (w/v) polysorbate 80, pH of 7.2, and 6 mg of RTH258. Another such formulation comprises 6.4% (w/v) or 5.8% sucrose, 12 mM or 10 mM sodium citrate, 0.02% (w/v) polysorbate 80, pH of 7.2, and 3 mg of RTH258. Preferred concentrations of RTH258 are about 120 mg/ml and about 60 mg/ml. Doses can be delivered, for example as 6 mg/50 μL and 3 mg/50 μL concentrations.

TABLE 1
Preferred Aqueous Formulation

| | | Concentration |
|---|---|---|
| | Concentration | Range |
| Component | (W/V %) | (W/V %) |
| RTH258 | 12 | 6-12 |
| Citric Acid, | 0.009 | 0.006-0.012 |
| anhydrous | | |
| Trisodium citrate | 0.4 | 0.2-0.6 |
| (dihydrate) | | |
| Sucrose | 6.75 | 4.5-11% |
| Polysorbate 80 | 0.02% | 0.01-0.05% |
| Hydrochloric acid or | pH 7.0 | pH 6.0-7.5 |
| Sodium hydroxide | | |
| Water for injection | qs 100 | qs 100 |

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLES

In the loading phase, treatment with RTH258 occurs every 6 weeks for five (5) consecutive injections (Day 0, Weeks 6, 12, 18 and 24).

The treatment interval during the maintenance phase is as follows:

From Week 24 onwards, patients receive one injection of RTH258 every 12 weeks. The patient is assessed for disease activity at Week 32, and every 12 weeks (e.g. Week 32, 36, 48, 60, 72, and 84) before or after getting a scheduled injection. If disease activity is identified at any of the assessments, the patient is assigned to receive treatment every 8 weeks (see Evaluation of Disease Activity below).

At Week 72, based on Disease Stability Assessment (see Assessment of Disease Stability below) the treatment provider has the option to extend the treatment interval by 4 Weeks, i.e. patients on q12w treatment schedule at Week 72 can be assigned to q16w and patients on q8w can be assigned to q12w. If the treatment provider identifies disease activity at a scheduled treatment visit (according to the patient specific treatment schedule q12w or q16w) the patient is assigned to q8w treatment schedule.

### Evaluation of Disease Activity:

The concept of the q12w/q8w regimen is to allocate patients according to their individual treatment needs to either a q12w or a q8w treatment schedule. The initial schedule is q12w and a patient will remain on q12w as long as the treatment provider does not identify DME disease activity requiring more frequent anti-VEGF treatment. Disease Activity Assessments (DAA) and a potential resulting adjustment of the treatment frequency are limited to pre-specified DAA-visits:
A more close monitoring of the patients individual treatment need takes place during the first q12w treatment interval with DAAs at Week 32 and 36 (i.e. for patients 8 and 12 weeks after the last loading injection) to make sure that patients with a high treatment need are identified early on
After the first q12w treatment interval DAA takes place together with the scheduled q12w treatment visits, e.g. at Week 48, Week 60, Week 72, Week 84, etc.

The treatment provider assesses DME disease activity to establish the patient's disease status at Week 28 (outcome of the loading treatment). The assessment of the disease activity is at the discretion of the treatment provider and should be made based on changes in vision and anatomical parameters with reference to the patients' disease status at Week 28. The outcome of this assessment is captured as:
‘q8w-need’: identified disease activity that according to the treatment provider requires more frequent anti-VEGF treatment, e.g.: ≥5 letters loss in BCVA (compared to Week 28) which, based on anatomical parameters, is attributable to DME disease activity.
‘no q8w-need’: otherwise if DAA reveals a need for more q8w treatment the subject is assigned to receive injections q8w thereafter. If disease status improves, the treatment provider can place the patient back on a q12w treatment schedule.

If DAA reveals a need for more frequent treatment, the patient is assigned to receive injections q8w thereafter, or up to a treatment interval extension based on the stability assessment at Week 72.

### Assessment of Disease Stability:

At Week 72, the treatment provider assesses a patient for the option to extend the current treatment interval by 4 weeks, i.e. to extend a q12w treatment schedule to q16w and q8w to q12w.

Based on the general concept that an extension of the treatment interval should only be considered for patients having shown sufficient disease stability under the current treatment schedule, the treatment provider will assess at Week 72 whether a 4-week extension of the treatment interval is adequate. The outcome of this assessment is captured as:
‘Extension of treatment interval’: according to the treatment provider there is sufficient disease stability to justify an extension of the treatment interval by 4 weeks, e.g. patient showed no disease activity during the two previous DAAs, i.e., at Week 60 and Week 72.
‘No extension of treatment interval’: otherwise patients not identified by the treatment provider for an extension of their treatment intervals continue with their latest treatment frequency considering adjustments according to future DAAs during each scheduled treatment visit.

### Activity Assessment

The following tests are performed to assess activity of RTH258 on visual function, retinal structure and leakage:
Best-corrected visual acuity with ETDRS-like chart at 4 meters
Anatomical markers on Optical Coherence Tomography
ETDRS DRSS score based on 7-field stereo Color Fundus Photography
Vascular leakage evaluation by Fluorescein Angiography

Visual acuity will be assessed at every treatment visit using best correction determined from protocol refraction (BCVA). BCVA measurements are taken in a sitting position using ETDRS—like visual acuity testing charts. The details of the procedure and training materials are provided in applicable manuals.

Optical Coherence Tomography (OCT) is assessed at screening (e.g., Day 0), and periodically during treatment visits. Treatment providers will evaluate the OCT to assess the status of disease activity. The OCT machine used for an individual patient should not change for the duration of the treatment. In addition to the standard OCT assessment, as optional assessment at sites that have the applicable equipment, OCT angiography should be done at baseline, Week 28, Week 52, Week 76, etc. If OCT angiography is performed, it should be done for a given patient from baseline. If OCT angiography is not performed at baseline, then it should not be introduced at later visits.

Color fundus photography and fluorescein angiography will be performed at screening, weeks 28, 52, and 76, etc. At sites that have the applicable equipment, optional wide-field angiography and fundus photography (at least 100 degrees) in study eye should be performed during the same visit, as the standard assessments (screening, weeks 28, 52, 76 and exit/premature discontinuation visit). Wide-field fundus photography does not replace 7-field color fundus photography images, hence both types of images must be taken. Wide-field images have to be collected from screening. If wide-field angiography and fundus photography were not taken at screening, then it should not be introduced at later visits.

Grading for Diabetic retinopathy severity scale (DRSS) will be performed by the treatment provider or a technician using criteria known to those of skill in the art.

BCVA as a measure of retinal function and OCT images to analyze anatomical changes are standard assessments to monitor DME and potential treatment effects in routine practice and clinical trials. Likewise established is FA that helps classifying the type of macular edema and is used to assess vascular leakage. Early Treatment Diabetic Retinopathy Study (ETDRS DRSS) is a recent addition to the tests conducted in clinical trials. This grading informs about the severity of the diabetic retinopathy underlying the macular edema.

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein. The claims should not be read as limited to the described order or elements unless stated to that effect. It should be understood that various changes in form and detail may be made without departing from the scope of the appended claims.

1. A method for treating diabetic macular edema (DME) in a patient, the method comprising:
a) administering to the patient five individual doses of a VEGF antagonist at 6-week intervals; and
b) administering to the patient an additional dose of the VEGF antagonist once every 8 weeks (q8w regimen) or once every 12 weeks (q12w regimen) thereafter.

2. The method of claim 1, further comprising assessing the patient for DME disease activity before or after administering every q8w or q12w dose.

3. The method of claim 2, wherein if worsening of DME disease activity is identified after a q12w dose, the patient is switched to a q8w regimen, wherein the additional doses are administered once every 8 weeks instead of once every 12 weeks.

4. The method of claim 3, wherein the worsening of DME disease activity is a loss of letters in best corrected visual acuity (BCVA), increased central subfield thickness (CST), and/or increased fluid accumulation compared to any previous assessment.

5. The method of claim 2, wherein at week 72 after the first dose was administered, the q12w treatment interval is extended by 4 weeks if the patient's DME disease activity is consistent over the previous two assessments.

6. The method of claim 3, wherein at week 72 after the first dose was administered, the q8w treatment interval is extended by 4 weeks if the patient's DME disease activity is consistent over the previous two assessments.

7. The method claim 3, wherein disease activity is assessed based on identifying dynamic changes in best corrected visual acuity (BCVA), central subfield thickness (CST), and/or intraretinal fluid status.

8. The method of claim 1, wherein the patient is a human.

9. The method of claim 1, wherein the anti-VEGF antagonist comprises the sequence of SEQ ID NO: 3.

10. (canceled)

11. The method of claim 9, wherein the concentration of the VEGF antagonist is about 60, 70, 80, 90, 100, 110, or 120 mg/ml.

12. A method for treating DME comprising administering to a patient five individual doses of a VEGF antagonist at 6-week intervals, followed by additional doses every 8 weeks (q8w regimen), wherein the VEGF antagonist is anti-VEGF antibody that comprises the variable light chain sequence of SEQ ID NO: 1 and the variable heavy chain sequence of SEQ ID NO: 2.

13. The method of claim 12, further comprising assessing the patient's DME disease activity before or after administering every q8w dose.

14. The method of claim 13, wherein if DME disease activity is improved relative to the previous assessment, the patient is switched to a q12w regimen, wherein the additional doses are administered once every 12 weeks instead of once every 8 weeks.

15. The method of claim 14, wherein at week 72 after the first dose was administered, the q12w treatment interval is extended by 4 weeks if the patient's DME disease activity is consistent over the previous two assessments.

16. The method of claim 15, wherein at week 72 after the first dose was administered, the q8w treatment interval is extended by 4 weeks if the patient's DME disease activity is consistent over the previous two assessments.

17. The method of claim 16, wherein disease activity is assessed based on identifying dynamic changes in best corrected visual acuity (BCVA), central subfield thickness (CST), and/or intraretinal fluid status.

18. The method of claim 12, wherein the patient is a human.

19. The method of claim 12, wherein the anti-VEGF antagonist is an antibody that comprises the sequence of SEQ ID NO: 3.

20. (canceled)

21. The method of claim 19, wherein the concentration of the VEGF antagonist is about 60, 70, 80, 90, 100, 110, or 120 mg/ml.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)


SEQUENCE LISTING (4 sequences)

SEQ ID NO: 1 - protein, 120 aa, Artificial Sequence
  Synthetic heavy chain variable domain
EVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 2 - protein, 111 aa, Artificial Sequence
  Synthetic light chain variable domain
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLG

SEQ ID NO: 3 - protein, 251 aa, Artificial Sequence
  Synthetic single-chain antibody
EIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS

SEQ ID NO: 4 - protein, 252 aa, Artificial Sequence
  Synthetic single-chain antibody
MEIVMTQSPSTLSASVGDRVIITCQASEIIHSWLAWYQQKPGKAPKLLIYLASTLASGVPSRFSGSGSGAEFTLTISSLQPDDFATYYCQNVYLASTNGANFGQGTKLTVLGGGGGSGGGGSGGGGSGGGGSEVQLVESGGGLVQPGGSLRLSCTASGFSLTDYYYMTWVRQAPGKGLEWVGFIDPDDDPYYATWAKGRFTISRDNSKNTLYLQMNSLRAEDTAVYYCAGGDHNSGWGLDIWGQGTLVTVSS